(19) United States
(12) Patent Application Publication
(10) Pub. No.: US 2023/0210375 A1
(43) Pub. Date: Jul. 6, 2023

(54) DEVICE FOR DIAGNOSING ABNORMALITY BY MEASURING MINIMAL CHANGE IN MUSCLE

(71) Applicants: KOREA INSTITUTE OF SCIENCE AND TECHNOLOGY, Seoul (KR); Korea University Research and Business Foundation, Seoul (KR)

(72) Inventors: Song Joo LEE, Seoul (KR); Jong Woo KANG, Seongnam-si (KR); Keun Tae KIM, Seoul (KR); Duguma Teshome Gemechu, Seoul (KR); Eunyoung SEO, Seoul (KR); Taehoon LEE, Seoul (KR)

(73) Assignees: KOREA INSTITUTE OF SCIENCE AND TECHNOLOGY, Seoul (KR); Korea University Research and Business Foundation, Seoul (KR)

(21) Appl. No.: 17/973,120

(22) Filed: Oct. 25, 2022

(30) Foreign Application Priority Data
Dec. 30, 2021 (KR) 10-2021-0192377

(51) Int. Cl. A61B 5/00; A61B 5/02; A61B 5/389

(52) U.S. Cl. CPC A61B 5/0051; A61B 5/4519; A61B 5/02007; A61B 5/7275; A61B 5/389; A61B 5/7264; A61B 5/746; A61B 5/6833; A61B 5/0022; A61B 2562/0247; A61B 2562/0219

(57) ABSTRACT

A device for diagnosing an abnormality by measuring a minimal change in a muscle according to an embodiment includes a vibration unit that provides vibration to a body part of a user; a measuring unit that detects a minimal change in a muscle by measuring a change in elasticity of the body part according to the vibration; and a processing unit that calculates an abnormality of the body part based on the change in the elasticity of the body part, wherein the processing unit calculates the abnormality of the body party by using an algorithm that detects a degree to which a specific value of the muscle is far from a distribution chart by analyzing data distribution or an anomaly detection algorithm that detects whether there is anomaly in a variable.

### CROSS REFERENCE TO RELATED APPLICATION

The present application claims priority to Korean Patent Application No. 10-2021-0192377, filed Dec. 30, 2021, the entire contents of which is incorporated herein for all purposes by this reference.

### BACKGROUND OF THE INVENTION

### Field of the Invention

The present invention relates to a device for diagnosing an abnormality by measuring a minimal change in muscle, and more particularly, to a device capable of providing vibration to a user's body part, and at the same time, calculating an abnormality of the body part based on a change in the muscle of the body part.

### Description of Government-Sponsored Research

This study was made with the support of a joint research project of KU Medicine-KIST TRC (Project title: Development of system for early diagnosis and monitoring of extremity deep vein thrombosis using sensor fusion technology, Project identification number: 2E3115J) under the supervision of the Korea Institute of Science and Technology and Korea University Ansan Hospital.

### Description of the Related Art

Deep vein thrombosis (DVT) is a disease that occurs due to blood clots in veins. This is a symptom in which venous blood in body parts (mainly in the lower extremities) stagnates when a person is in an immobile position for a long time or is in various situations where blood clots are likely to occur, causing blood clots to form in deep veins. It is also called ‘economy class syndrome’ because it is a disease that often occurs in passengers sitting in narrow seats during long-distance flights. If left untreated, deep vein thrombosis can cause limb necrosis, pulmonary embolism, and breathing difficulties, leading to death, so it is important to diagnose these symptoms early and prevent the condition from getting worse.

Conventionally, deep vein thrombosis was diagnosed mainly by examining a patient's body through ultrasound or through blood tests. Such a diagnosis method may be effective in a situation where a patient can receive periodic ultrasound or blood tests in a hospital, clinic, etc., but early diagnosis is difficult if it is caused by a long flight or driving. Preemptive methods such as mechanical prophylaxis (compression stockings, air compression devices, etc.) or pharmacological prophylaxis (administration of anticoagulants, etc.) exist, but in many cases, practical application is difficult, resulting in a large number of patients with deep vein thrombosis every year.

### Documents of Related Art

(Patent Document 1) KR Patent Laid-Open Publication No. 10-2010-0049382

### SUMMARY OF THE INVENTION

Accordingly, an object of the present invention is to provide a diagnosis device capable of not only calculating an abnormality of a body part based on a change in muscle of a body part, but also diagnosing deep vein thrombosis (DVT) at an early stage.

A device for diagnosing an abnormality by measuring a minimal change in a muscle according to an embodiment includes a vibration unit that provides vibration to a body part of a user; a measuring unit that detects a minimal change in a muscle by measuring a change in elasticity of the body part according to the vibration; and a processing unit that calculates an abnormality of the body part based on the change in the elasticity of the body part, wherein the processing unit calculates the abnormality of the body party by using an algorithm that detects a degree to which a specific value of the muscle is far from a distribution chart by analyzing data distribution or an anomaly detection algorithm that detects whether there is anomaly in a variable.

According to an embodiment, the processing unit may calculate a risk of thrombosis based on the abnormality of the body part.

According to an embodiment, the device may further include a display unit that displays the risk of thrombosis.

According to an embodiment, the device may further include a control unit that controls an operation of the vibration unit.

According to an embodiment, the vibration unit may receive a control signal from the control unit and provide the vibration of a specific frequency through a vibrator attached to the body part.

According to an embodiment, the device may further include a pressure sensor positioned between the vibrator and the body part of the user to sense pressure according to the vibration, wherein the processing unit may be configured to indicate the pressure according to the vibration through the display unit.

According to an embodiment, the measuring unit may include an accelerometer that detects reflex vibration generated in the body part in response to the vibration.

According to an embodiment, the measuring unit may include an electromyograph that measures an electromyograph signal of the body part.

According to an embodiment, the processing unit may obtain an approximate function of each of measurement values of reflex vibration and electromyograph signal of the body part using a distribution of Bayesian probability value based on a change in the measurement values of the reflex vibration and EMG signal of the body part over time, and may convert each of the measurement values of the reflex vibration and EMG signal of the body part into a value within a range of 0 to 1 by using the calculated approximate function.

According to an embodiment, an anomaly detection algorithm used by the processing unit may include an isolation forest algorithm that detects anomaly by splitting anomaly data based on a tree.

According to an embodiment, the display unit may numerically indicate the risk of thrombosis through a display device.

According to an embodiment, the display unit may be configured to turn on an LED element when the risk of thrombosis is greater than or equal to a threshold.

According to an embodiment, the device may be implemented to be detachable as a wearable patch.

According to an embodiment, the processing unit may be connected to wirelessly communicate with the display unit using one or more of radio frequency (RF), Wi-Fi, cellular, Bluetooth, Bluetooth Low Energy (BLE), personal area network (PAN), short-wavelength UHF, and a combination thereof.

According to an embodiment of the present invention, a devise for diagnosing an abnormality of a body part at an early stage by providing vibration of a specific frequency using a vibrator attached to the user's body, and measuring the reflex vibration and EMG signal of the muscle sensed in response to the vibration.

The diagnosis device according to an embodiment determines that the occurrence of deep vein thrombosis is high when an abnormal change appears in the measurement value of reflex vibration measured by an accelerometer/or the measurement value of EMG signal measured by an electromyography, and notifies the user of abnormalities in the body parts or risk of thrombosis in real time.

The diagnosis apparatus according to an embodiment includes a vibrator attached to a body part and a sensor (accelerometer, electromyography, etc.) capable of detecting a change in the body part, so that it is easy to install and use. For example, the risk of thrombosis may be measured at regular intervals using the vibrator and sensor provided under an airplane seat. Therefore, it is possible to diagnose deep vein thrombosis earlier than the conventional ultrasound or blood test methods.

### BRIEF DESCRIPTION OF THE DRAWINGS

FIG. 1 is a block diagram illustrating a concept of a devise for diagnosing an abnormality by measuring a minimal change in a muscle according to an embodiment.

FIGS. 2A to 2D illustrate a process in which a processing unit calculates a degree to which the reflex vibration or EMG signal of a body part moves away from a data distribution by using a Bayesian algorithm according to an embodiment.

FIGS. 3A to 3C illustrate a process in which a processing unit calculates an abnormality of the reflex vibration or EMG signal of a body part using an anomaly detection algorithm according to an embodiment.

FIG. 4 illustrates a device for diagnosing an abnormality that is implemented as a wearable patch and measures an abnormality by measuring a minimal change in a muscle according to an embodiment.

FIG. 5 illustrates a process in which a processing unit calculates a risk of thrombosis and a display unit warns a user through wireless communication, according to an embodiment.

### DETAILED DESCRIPTION OF THE INVENTION

The terms used in the present specification have been selected as widely used general terms as possible while considering their functions, but may vary depending on the intention or custom of those skilled in the art or the advent of new technology. In addition, in a specific case, there is a term arbitrarily selected by the applicant, and in this case, the meaning will be described in the description of the corresponding specification. Therefore, it is intended to clarify that the terms used in this specification are not simply names of terms, but should be interpreted based on the actual meaning of the terms and the contents of the entire specification.

Hereinafter, the embodiments will be described in detail with reference to the accompanying drawings and the contents described in the accompanying drawings, but the scope of the claims is not limited or restricted by the embodiments.

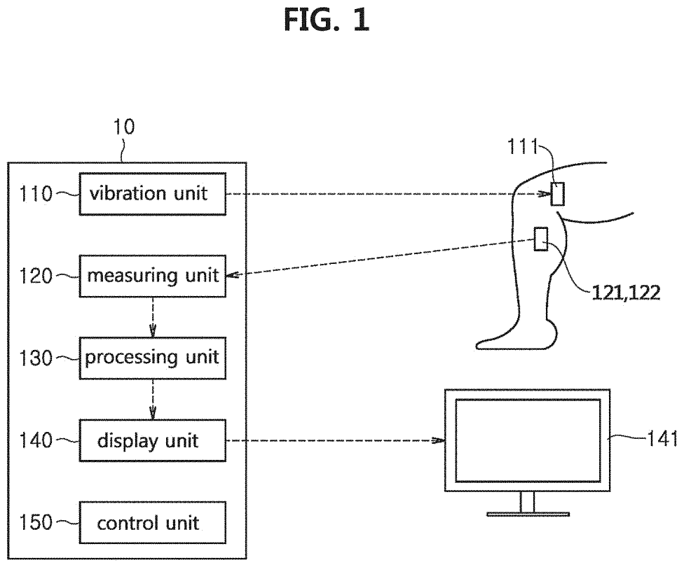

FIG. 1 is a block diagram illustrating a concept of a device for diagnosing an abnormality by measuring a minimal change in a muscle according to an embodiment. Referring to FIG. 1, a device for diagnosing an abnormality by measuring a minimal change in a muscle 10 (hereinafter, “diagnosis device”) according to an embodiment is configured to include a vibration unit 110, a measuring unit 120, and a processing unit 130, a display unit 140, and a control unit 150.

Although the above components are shown separately for the convenience of conceptual description, they do not necessarily have to be implemented as independent devices or programs. For example, each of the components may be implemented by one processing unit or program, or may be implemented by an organic combination of two or more independent processing units or programs.

The vibration unit 110 receives a control signal from the control unit 150 and provides the vibration of a specific frequency through a vibrator 111 attached to a body part. The vibration unit 110 receives the control signal, for example, an input for the intensity, frequency, and form of vibration to be provided to the user (regular intensity or periodically repeating strength and weakness) from the control unit 150, and transmit it to the vibration unit 110 by wire or wirelessly.

The vibrator 111 is a device having a power device such as a motor to convert electrical energy into physical vibration, and is not limited to a specific shape or size. The vibrator 111 may be connected to the diagnosis device 10 by wire or through a wireless network (Bluetooth, WiFi, infrared communication, etc.). In addition, the vibrator 111 may be powered by a motor by wire, or may be powered by a built-in battery. In one embodiment, the vibrator 111 may be attached to the user's body part (arm, leg, etc.) through a fixing part such as a strap or adhesive tape that can be worn on the user's body part.

According to an embodiment, a pressure sensor (not shown) for sensing pressure according to vibration between the vibrator 111 and the user's body part may be further provided. The processing unit 130 to be described later may display a change in pressure according to the vibration on the display unit 140. This allows the user to know in real time whether the vibrator is providing vibration at the appropriate pressure level. Accordingly, when the pressure is too low or too high, the operations of the vibration unit 110 and vibrator 111 may be controlled through the control unit 150.

The measuring unit 120 may measure a change in elasticity of a body part according to the vibration provided by the vibration unit 110 to detect a minimal change in a muscle. For example, when deep vein thrombosis (DVT) occurs, blood clots are formed in the vein, which causes muscle stiffness, and the measuring unit 120 measures a change in elasticity of a muscle to detect these symptoms.

The measuring unit 120 detects a change in elasticity of a body part through an accelerometer 121 and/or an electromyography 122, and warns the user by calculating an abnormality or risk of thrombosis of the body part when an abnormal change occurs.

According to an embodiment, the measuring unit 120 may sense the reflex vibration generated in the user's body part through an accelerometer 121. Reflex vibration refers to the tremor that occurs in the surrounding muscles when an arbitrary vibrational stimulus is applied to the muscle. In general, in an environment with the same muscle quality or blood flow, when the vibration stimulation of the same characteristic (intensity, frequency, stimulation interval) is applied, the reflex vibration of the corresponding characteristic appears at a constant level. In contrast, when deep vein thrombosis occurs and the muscle becomes stiff or the blood flow changes, the characteristic of the reflex vibration sensed in the muscle changes rapidly. The accelerometer 121 is a device for converting a minimal movement of a muscle into an electrical signal, and detects a change in vibration characteristics due to the muscular dystrophy or thrombosis and transmits it to the processing unit 130. After the development of muscular dystrophy or deep vein thrombosis, the measured value of measured reflex vibration changes significantly.

According to an embodiment, the measuring unit 120 may measure an electromyograph (EMG) signal of the user's body part through an electromyograph. The electromyograph is a device that records electrical activity according to muscle contraction using the electrodes attached to or inserted into the body parts. As with the above-mentioned reflex vibration, when deep vein thrombosis occurs and the muscle is stiff or there is a change in blood flow, the EMG signal also changes. The EMG 122 detects a change in the EMG signal and transmits it to the processing unit 130. After the development of deep vein thrombosis, the measured value of measured EMG signal changes significantly.

The processing unit 130 calculates an abnormality of a body part based on a change in elasticity of the body part. The processing unit 130 may calculate an abnormality of a body part by using an algorithm that analyzes data distribution to detect a degree to which a specific value of a muscle moves away from the distribution or an anomaly detection algorithm that detects whether there is anomaly in a variable. In an embodiment, the abnormality of the body part calculated by the processing unit 130 may mean a risk of thrombosis.

The processing unit 130 combines and simultaneously uses the measurement results of reflex vibration and EMG signal (in this case, each measurement result may be given a weight), or uses each result independently to calculate an abnormality of muscle or a risk of thrombosis.

The display unit 140 displays the risk of thrombosis calculated by the processing unit 130 to the user through an external device. According to an embodiment, the display unit 140 may display the calculation result as a numerical value on a display device 141 (refer to FIG. 1). According to another embodiment, the display unit 140 may warn the user by turning on an LED element (not shown) when the abnormality of muscle or the risk of thrombosis is greater than or equal to a threshold (e.g., if the risk of deep vein thrombosis is high, a red light is turned on, and if the risk is low, a green light is turned on).

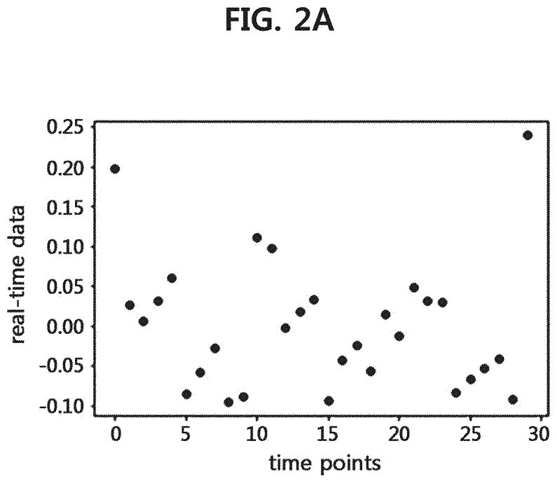

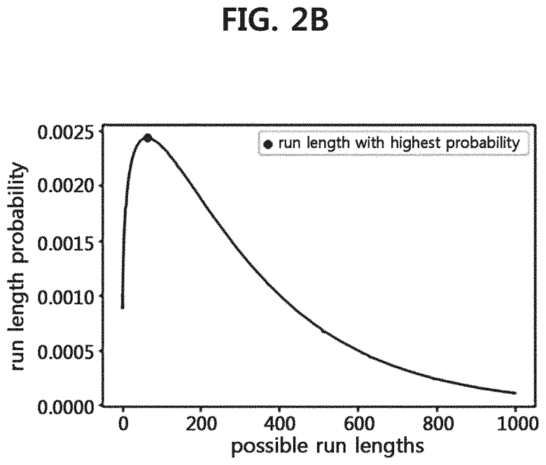

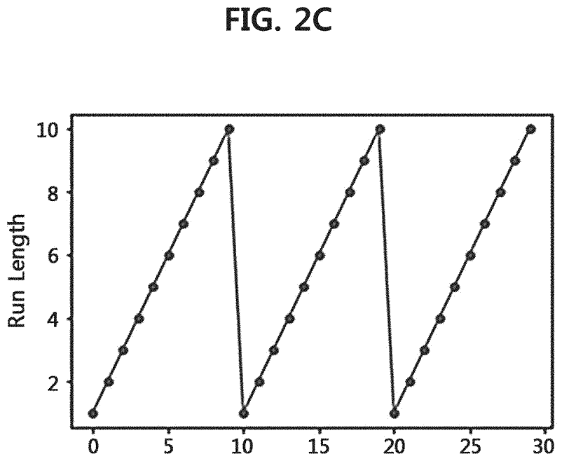

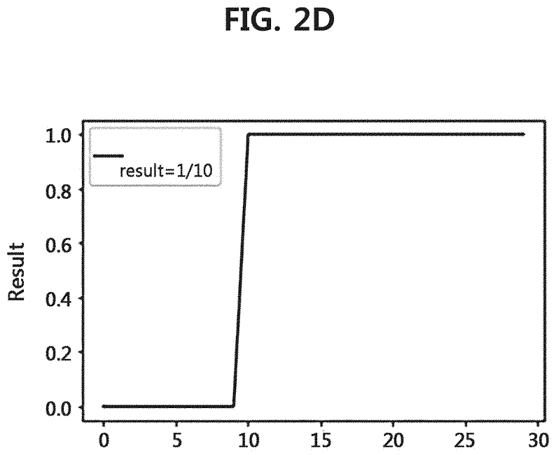

FIGS. 2A to 2D illustrate a process in which the processing unit calculates a degree to which the reflex vibration or EMG signal of a body part moves away from data distribution by using a Bayesian algorithm according to an embodiment.

Referring to FIGS. 2A to 2D, the processing unit 130 may calculate the abnormality of the body part using an algorithm that detects the degree to which a specific value of muscle has moved away from the distribution chart by analyzing the data distribution of the measurement result of the reflex vibration of the body part and the measurement result of the EMG signal. The algorithm may follow the distribution of Bayesian probability values. The processing unit 130 obtains an approximate function for each of the measurement values of the reflex vibration and the EMG signal of the body part based on a change in the measurement values of the reflex vibration and EMG signal of the body part over time, and converts each of the measurement values of the reflex vibration and EMG signal of the body part into a value within a range of 0 to 1 using the obtained approximate function. Here, the measurement value of reflex vibration or EMG signal of the body part converted to a value within the range of 0 to 1 may be a value indicating the probability that the thrombosis will be measured when a muscular dystrophy or thrombosis occurs in a patient, but the embodiment is not limited thereto.

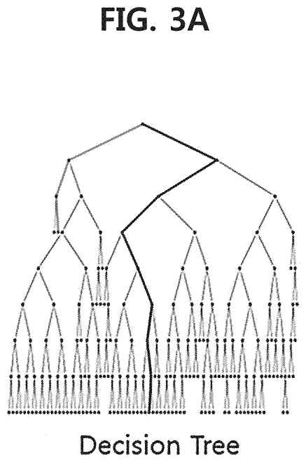

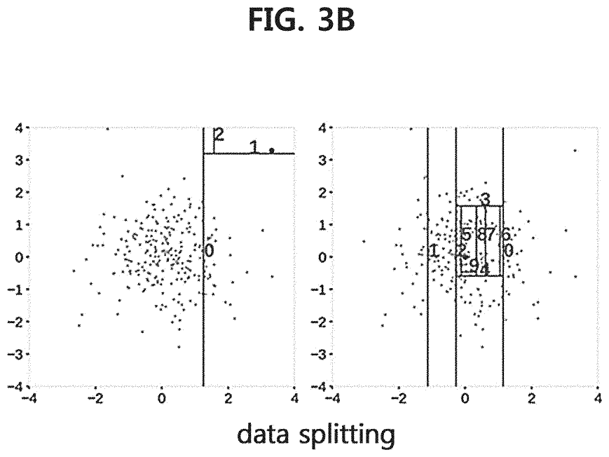

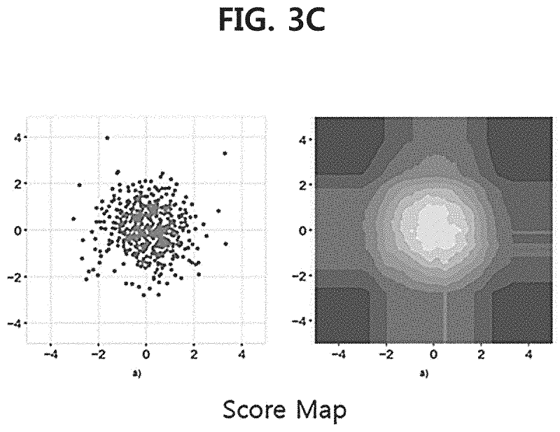

FIGS. 3A to 3C illustrate a process in which the processing unit 130 calculates an abnormality of reflex vibration or EMG signal of a body part using an anomaly detection algorithm according to an embodiment.

Referring to FIGS. 3A to 3C, the processing unit 130 can calculate an abnormality in reflex vibrations or EMG signals of a body part by using an isolation forest algorithm that detects an anomaly by isolating anomaly data based on a tree. The anomaly detection refers to detecting data that is significantly different from the majority of data or unique data. Here, anomaly may be expressed as noise, deviation, or exception. The anomaly detection is to find outlier, which is data showing different pattern in the collected data. The isolation forest technique expresses the data set in the form of a decision tree, and follows the depth direction of the decision tree in the case of splitting a normal value and uses the characteristic of splitting at the top of the decision tree in the case of splitting outlier. Using these characteristics, the isolation forest makes it possible to split normal values and outliers based on how many times the decision tree is descended and split. If there is a sudden change in the frequency component of the reflex vibration measured by the accelerometer and/or the frequency component of the EMG signal measured by the EMG, it is split as outlier. The greater the number of splitting, the higher the possibility of muscular dystrophy or deep vein thrombosis.

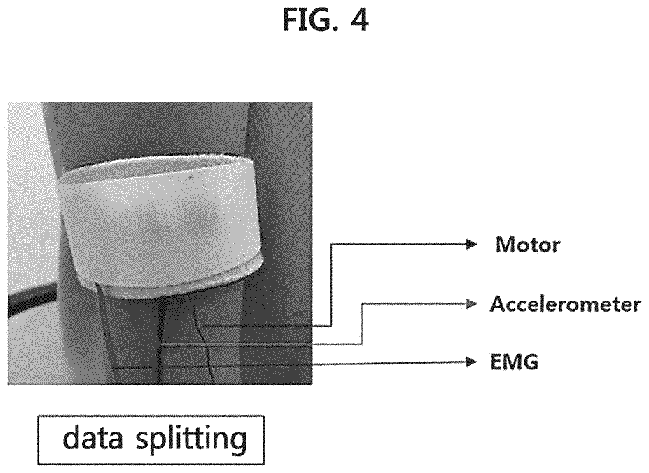

FIG. 4 illustrates a device for diagnosing abnormality that is implemented to be detachable as a wearable patch and measures a minimal change in a muscle, according to an embodiment.

Referring to FIG. 4, many wearable devices using IoT technology have recently appeared. Due to the characteristics of the wearable device, it should be easy to carry, and it should be light and capable of operating for a long time. A small device such as a wearable device is used in a form that is in direct contact with a person's body or is attached to clothes or other accessories without direct contact with the body.

In particular, among wearable devices, a wearable multi-biosignal measuring device such as a wearable electromyography device is a device that uses a sensor such as a patch-type electrode to form a contact point with various body parts (chest, wrist, ankle, etc.) of a subject to measure biosignals such as electromyography. This device is used to predict or diagnose an abnormality in body parts or the occurrence of diseases such as thrombosis by monitoring biosignals.

By implementing the diagnostic device of the present invention in a detachable manner as a wearable patch, it is possible to easily monitor bio-signals such as electromyography in daily life. Thus, medical personnel can be continuously provided with the patient's condition through the motor unit, measuring unit, and processing unit mounted on the wearable patch, and can rapidly diagnose and treat predicted disease, thereby reducing the risk of death.

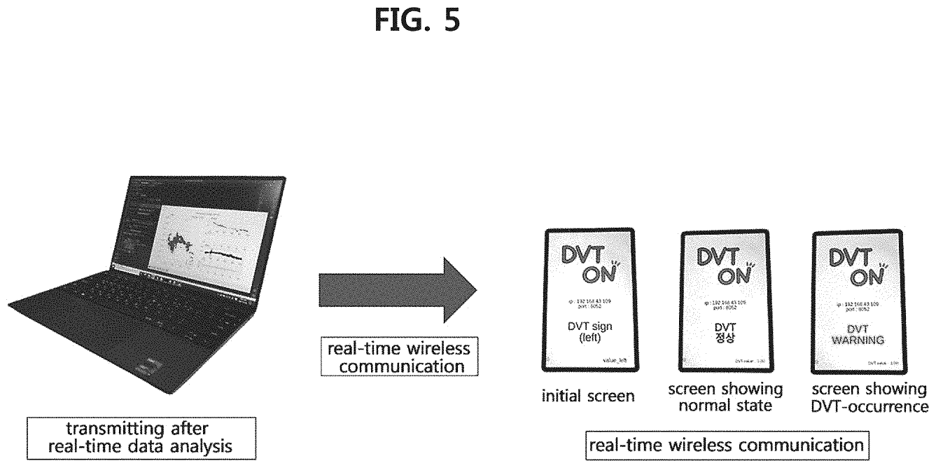

FIG. 5 illustrates a process in which the processing unit 130 calculates an abnormality of muscle or a risk of thrombosis, and a display unit warns a user through wireless communication, according to an embodiment.

The processing unit 130 may be connected to communicate wirelessly with the display unit 140 using one or more of radio frequency (RF), Wi-Fi, cellular, Bluetooth, Bluetooth Low Energy (BLE), personal area network (PAN), short-wavelength UHF, and a combination thereof.

Referring to FIG. 5, the display unit 140 displays the risk of thrombosis calculated by the processing unit 130 to the user through an external device. According to an embodiment, the display unit 140 may display the calculation result as a numerical value on the display device 141. According to another embodiment, the display unit 140 may warn the user by turning on an LED element (not shown) when the risk of thrombosis is greater than or equal to a threshold (e.g., if the risk of deep vein thrombosis is high, a red light is turned on, and if the risk of deep vein thrombosis is low, a green light is turned on).

According to the diagnostic device described above, the vibration of a specific frequency is provided using the vibrator attached to the user's body, and a muscular dystrophy or deep vein thrombosis is diagnosed by measuring the reflex vibration and EMG signal of the muscle sensed in response to the vibration. The diagnosis device according to an embodiment is configured to include the vibrator attached to a body part and the sensor capable of sensing a change in the body part, so that it is easy to install and use. For example, by using the vibrator and sensor provided under an airplane seat, it is possible to automatically measure and warn an abnormality of the body part or a risk of thrombosis of a traveler. According to this, it is possible to diagnose muscular dystrophy and deep vein thrombosis at an earlier stage compared to the conventional ultrasound examination method or blood test method.

Although the above has been described with reference to the embodiments, it will be understood by those skilled in the art that various modifications and changes can be made in the present invention without departing from the spirit and scope of the present invention as set forth in the following claims.

What is claimed is:

1. A device for diagnosing an abnormality by measuring a minimal change in a muscle, comprising,
a vibration unit that provides vibration to a body part of a user;
a measuring unit that detects a minimal change in a muscle by measuring a change in elasticity of the body part according to the vibration; and
a processing unit that calculates an abnormality of the body part based on the change in the elasticity of the body part,
wherein the processing unit calculates the abnormality of the body party by using an algorithm that detects a degree to which a specific value of the muscle is far from a distribution chart by analyzing data distribution or an anomaly detection algorithm that detects whether there is anomaly in a variable.

2. The device according to claim 1, wherein the abnormality of the body part calculated by the processing unit is a risk of thrombosis.

3. The device according to claim 1, further comprising a display unit that displays the abnormality of the body part.

4. The device according to claim 1, further comprising a control unit that controls an operation of the vibration unit.

5. The device according to claim 4, wherein the vibration unit receives a control signal from the control unit and provides the vibration of a specific frequency through a vibrator attached to the body part.

6. The device according to claim 5, further comprising a pressure sensor positioned between the vibrator and the body part of the user to sense pressure according to the vibration,
wherein the processing unit is configured to indicate the pressure according to the vibration through the display unit.

7. The device according to claim 1, wherein the measuring unit includes an accelerometer that detects reflex vibration generated in the body part in response to the vibration.

8. The device according to claim 1, wherein the measuring unit includes an electromyograph that measures an electromyograph signal of the body part.

9. The device according to claim 1, wherein the processing unit obtains an approximate function of each of measurement values of reflex vibration and electromyograph signal of the body part using a distribution of Bayesian probability value based on a change in the measurement values of the reflex vibration and EMG signal of the body part over time, and converts each of the measurement values of the reflex vibration and EMG signal of the body part into a value within a range of 0 to 1 by using the calculated approximate function.

10. The device according to claim 1, wherein an anomaly detection algorithm used by the processing unit includes an isolation forest algorithm that detects anomaly by splitting anomaly data based on a tree.

11. The device according to claim 3, wherein the display unit displays numerically indicates the abnormality of the body part through a display device.

12. The device according to claim 3, wherein the display unit is configured to turn on an LED element when the abnormality of the body part is greater than or equal to a threshold.

13. The device according to claim 1, wherein the device is detachable as a wearable patch.

14. The device according to claim 3, wherein the processing unit is connected to wirelessly communicate with the display unit using one or more of radio frequency (RF), Wi-Fi, cellular, Bluetooth, Bluetooth Low Energy (BLE), personal area network (PAN), short-wavelength UHF, and a combination thereof.
